(19) United States
(12) Patent Application Publication
(10) Pub. No.: US 2015/0105710 A1
(43) Pub. Date: Apr. 16, 2015

(54) UPPER BODY POSTURE CORRECTOR ASSEMBLY

(71) Applicant: Alicia CONSTAIN, Miami, FL (US)

(72) Inventor: Alicia CONSTAIN, Miami, FL (US)

(21) Appl. No.: 14/052,855

(22) Filed: Oct. 14, 2013

(51) Int. Cl. A61F 5/02

(52) U.S. Cl. CPC A61F 5/026
USPC 602/19

(57) ABSTRACT

The present invention pertains to a posture correcting assembly comprising a first strap with a first loop, a first band and a first attachment point; and a second strap with a second loop, a second band and a second attachment point, wherein the first loop hangs from a first shoulder of the user's body, the second loop hangs from a second shoulder of the users body, the first band wraps posteriorly around the user's body, the second band wraps posteriorly around the user's body, and the first attachment point and the second attachment point interconnect at the anterior side of the user's body. The present invention also pertains to a method for placing the fore mentioned posture correcting assembly on a user's body.

### FIELD OF THE INVENTION

The present invention relates to the art of body posture correction devices.

### CROSS REFERENCE TO RELATED APPLICATIONS

Not applicable.

### STATEMENT REGARDING FEDERALLY-SPONSORED RESEARCH AND DEVELOPMENT

Not applicable.

### BACKGROUND OF THE INVENTION

Many health problems can be traced back to poor body posture. Bodies in their most efficient posture experience better circulation, more effective respiration and, as a result, better distribution of the nutrients your body needs. Poor posture, on the other hand, causes fatigue which leads to chronic aches and pains. Lower backs are a key structure in our bodies, these symptoms often manifest as back pain. The biggest cause of poor posture is muscles that are either weak, or tensed to the point of causing fatigue.

One issue is that postures feel completely natural to a person, even if told that they are bad and unhealthy. Poor posture is a quite common issue, and can be seen to some extent in almost anyone.

There is are way to address problems of poor posture, namely, by using a back brace or other posture corrector to provide muscles with the support they need. By redirecting the posture to something more efficient, the fatigue toxins accumulated during regular daily activities will slowly vanish and take the pain along with them. Posture correctors provide the body the support it requires in a way that demands little effort.

Unfortunately, posture correctors have many problems ultimately making them a highly unsuitable solution for almost all postural problems. For example, the arm straps are usually very tight and press hard upwards against the armpits and cut off circulation to the arms. The material is also usually rough so there's a burning feeling wherever there's pressure, and it leaves marks on the skin after wearing even only for a few minutes. The most prominent issue is that they are uncomfortable and people do not want to wear them for long periods of time. Usually the shoulders and back becomes irritated because the corrector does not follow the natural contour of a human body.

### SUMMARY OF THE INVENTION

The present invention depicts an inventive solution to the fore mentioned issues related to straightening of the human back and wearing a comfortable back-support.

With the foregoing and other objects in view, there is provided, in accordance with the invention, a back support apparatus including: a left strap that includes: a left-first-loop, a left-second-loop, a left-belt-band and a left-attachment-point. The back support apparatus further includes a right strap that includes: a right-first-loop, a right-second-loop, a right-band and a right-attachment-point.

The present invention pertains to a posture correcting assembly comprising a first strap with a first loop, a first band and a first attachment point; and a second strap with a second loop, a second band and a second attachment point, wherein the first loop hangs from a first shoulder of the user's body, the second loop hangs from a second shoulder of the users body, the first band wraps posteriorly around the user's body, the second band wraps posteriorly around the user's body, and the first attachment point and the second attachment point interconnect at the anterior side of the user's body. The present invention also pertains to a method for placing the fore mentioned posture correcting assembly on a user's body.

In at least one embodiment, the present invention pertains to a posture correcting assembly comprising a posture correcting assembly, including: a left strap defining a left loop surrounding a left shoulder, the left strap further including: a left-belt-band wrapping posteriorly around a user's body; and a left-attachment-point; and a right strap defining a right loop surrounding a right shoulder, the right strap further including: a right-belt-band wrapping posteriorly around a user's body; and a right-attachment-point interconnect and is demountably disposed the left-attachment-point at the anterior side of a user's body.

In some embodiments, the left strap and the right strap may be constructed of a combination of elastic, semi-elastic, and rigid materials. In some embodiments, the left strap and the right strap may be constructed of a combination of elastic and semi-elastic materials. In some embodiments, the left strap and the right strap may be constructed of a combination of elastic and rigid materials. In some embodiments, the left strap and the right strap may be constructed of a combination of semi-elastic and rigid materials.

Unless otherwise defined, all terms of art, notations and other scientific terms or terminology used herein are intended to have the meanings commonly understood by those of skill in the art to which this invention pertains. In some cases, terms with commonly understood meanings are defined herein for clarity and/or for ready reference, and the inclusion of such definitions herein should not necessarily be construed to represent a substantial difference over what is generally understood in the art. Many of the techniques and procedures described, or referenced herein, are well understood and commonly employed using conventional methodology by those skilled in the art. As appropriate, procedures involving the use of commercially available kits and reagents are generally carried out according to manufacturer defined protocols and/or parameters, unless otherwise noted.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Other elements may optionally be present other than the elements specifically identified by the “and/or” clause, whether related or unrelated to those elements specifically identified unless clearly indicated to the contrary. Thus, as a non-limiting example, a reference to “A and/or B,” when used in conjunction with open-ended language such as “comprising” can refer, in one embodiment, to A without B (optionally including elements other than B); in another embodiment, to B without A (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein in the specification and the claims, “interconnection,” “interconnect,” “interconnects” means to put or bring together so as to form a unit catenate, to chain, to couple, to hook, to connect, to interlink, to join, link, by using interacting parts mating demountably disposed into each-other.

As used herein, the term “demountably disposed” and “demountably disposes” refers to the placement of two faces wherein the two faces can be removed and placed back together with little effort many times. As a matter of example and in no means to be used as a limit to the words “demountably disposed” and “demountably disposes” comprises the use of; double sided tape, adhesives, magnetics, hooks, loops buttons, snap buttons, zippers, VELCRO or any combination thereof.

### BRIEF DESCRIPTION OF THE SEVERAL VIEWS OF THE DRAWINGS

The foregoing summary, as well as the following detailed description of the invention, will be better understood when read in conjunction with the appended drawings. For illustrating the invention, the figures are shown in the embodiments that are presently preferred. It should be understood, however, that the invention is not limited to the precise arrangements and instrumentalities shown. In the drawings:

FIG. 1 is a side view showing the posture correcting assembly, that is at least one embodiment of the present invention that includes: a left strap, and matching right strap.

FIG. 2 is a side view of the right strap of the posture correcting assembly shown in FIG. 1, as it is worn by a user.

FIG. 3 is a frontal view showing one embodiment of the posture correcting assembly while being worn by a user.

FIG. 4 is a view of the back of user wearing the posture correcting assembly, where both the left strap and the right strap cross and overlap about the lower back of a user's body.

FIG. 5 is frontal view of a user wearing an alternative embodiment of the posture correcting assembly, where the right strap includes a single right-first-loop looping around right shoulder and the left strap contains a single left-first-loop looping around left shoulder.

FIG. 6 is a graphical depiction of the back of a user wearing the posture correcting assembly further including at least one left-vertical-band.

FIG. 7 is a graphical depiction of the back of a user wearing the posture correcting assembly showing adjustable left-vertical-band and adjustable horizontal band.

FIG. 8 shows an exploded view of the left-vertical-band band of the posture correcting assembly FIG. 7.

FIG. 9 shows an exploded view of the horizontal band of the posture correcting assembly FIG. 7.

FIG. 10 are four graphical representations and multiple views, namely A, B, C and D, of an embodiment of the posture correcting assembly as worn by a user.

FIG. 11 are four graphical representations and multiple views, namely A, B, C and D, of an embodiment of the posture correcting assembly as worn by a user.

FIG. 12 are three sequential graphical representations, namely A, B, and D, depicts a method of using the posture correcting assembly.

### DETAILED DESCRIPTION OF THE INVENTION

While the specification concludes with claims defining the features of the invention that are regarded as novel, it is believed that the invention will be better understood from a consideration of the following description in conjunction with the drawing figures, in which like reference numerals are carried forward. It is to be understood that the disclosed embodiments are merely exemplary of the invention, which can be embodied in various forms.

The present invention provides a novel and bodily positioning apparatus with back support that provide instant back restraint. Embodiments of the invention provide a foldable bodily posture correcting assembly, with a left strap, and matching right strap, to give a user instant relief for back pain. In addition, embodiments of the invention provide a method to correct a user's posture.

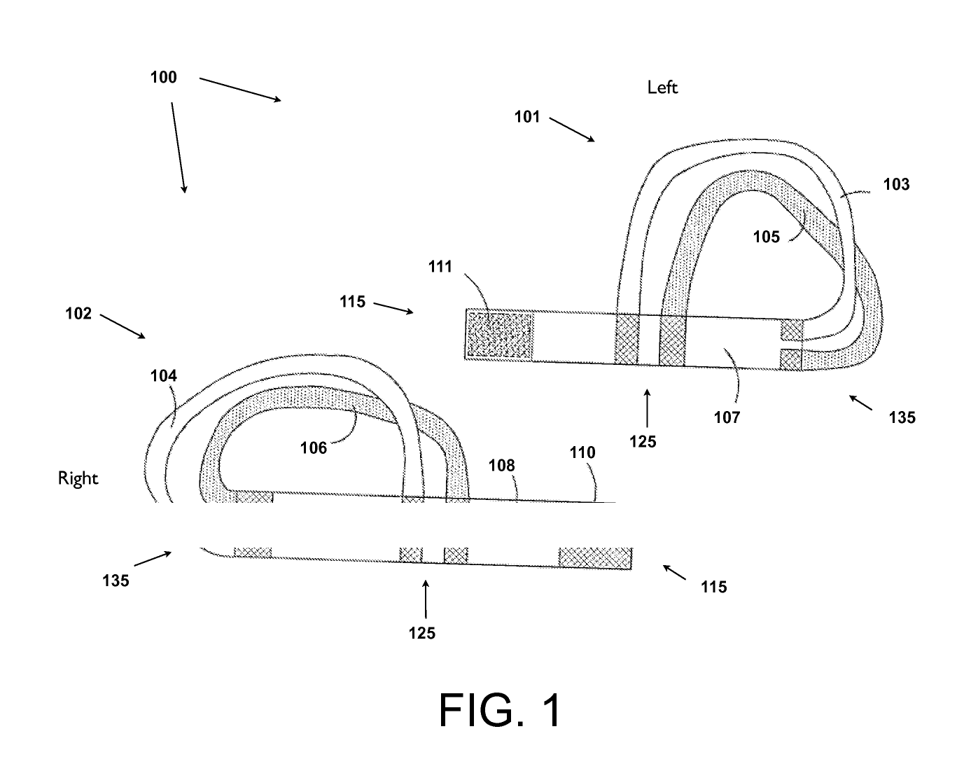

Referring now to the drawings in detail, FIG. 1 is a side view showing the posture correcting assembly 100. The posture correcting assembly 100 shown in FIG. 1 is at least one embodiment of the present invention that includes: a left strap 101, and matching right strap 102. The left strap includes: a left-first-loop 103, a left-second-loop 105, a left-belt-band 107 and a left-attachment-point 111. The right strap 102, includes: a right-first-loop 104, a right-second-loop 106, a right-belt-band 108 and a right-attachment-point 110. Both left-belt-band 107 and right-belt-band 108, comprise a first end 115, a middle portion 125, and a second end 135.

The left-first-loop 103, and the left-second-loop 105 are fixedly attached to the second end 135 of left-belt-band 107 and loop back and are fixedly attached individually at the a middle portion 125 left-belt-band 107. Similarly, the right-first-loop 104, and the right-second-loop 106 are fixedly attached to the second end 135 of right-belt-band 108 and loop back and are fixedly attached individually at the a middle portion 125 right-belt-band 108.

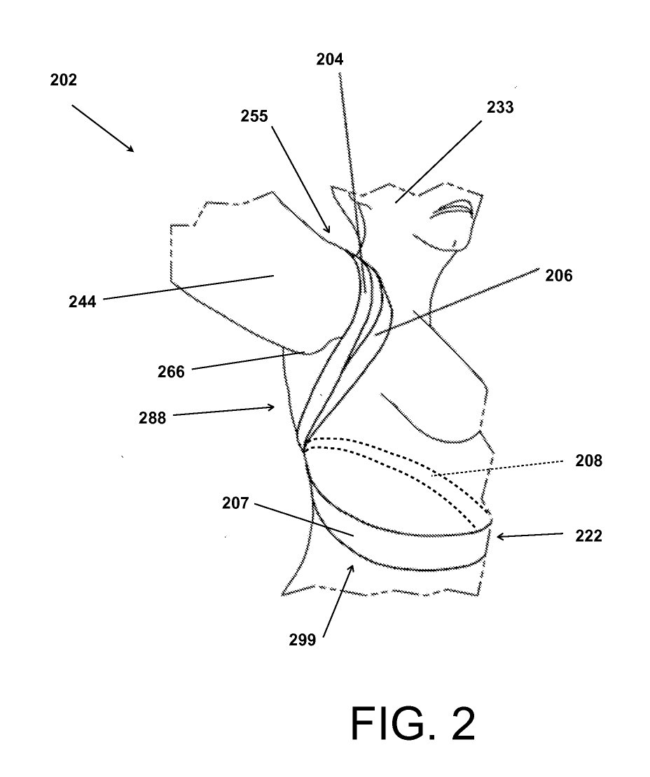

FIG. 2 is a side view of the right strap 202 of the posture correcting assembly 100 shown in FIG. 1, as it is worn by a user 233. Here, the right strap 202 is worn by placing the right-first-loop 104, and the right-second-loop 106 into a user's 233 arm 244 and around a user's 233 shoulder 255 and under user's 233 armpit 266. The right-band 108 (hidden lines) goes around a user's 233 back 288 and waist 299 to the abdomen 222 in front of the user to couple with left-belt-band 207. It is intended for both left and right attachment points 110 and 111 (shown in FIG. 1) to demountably mate together about abdomen 222.

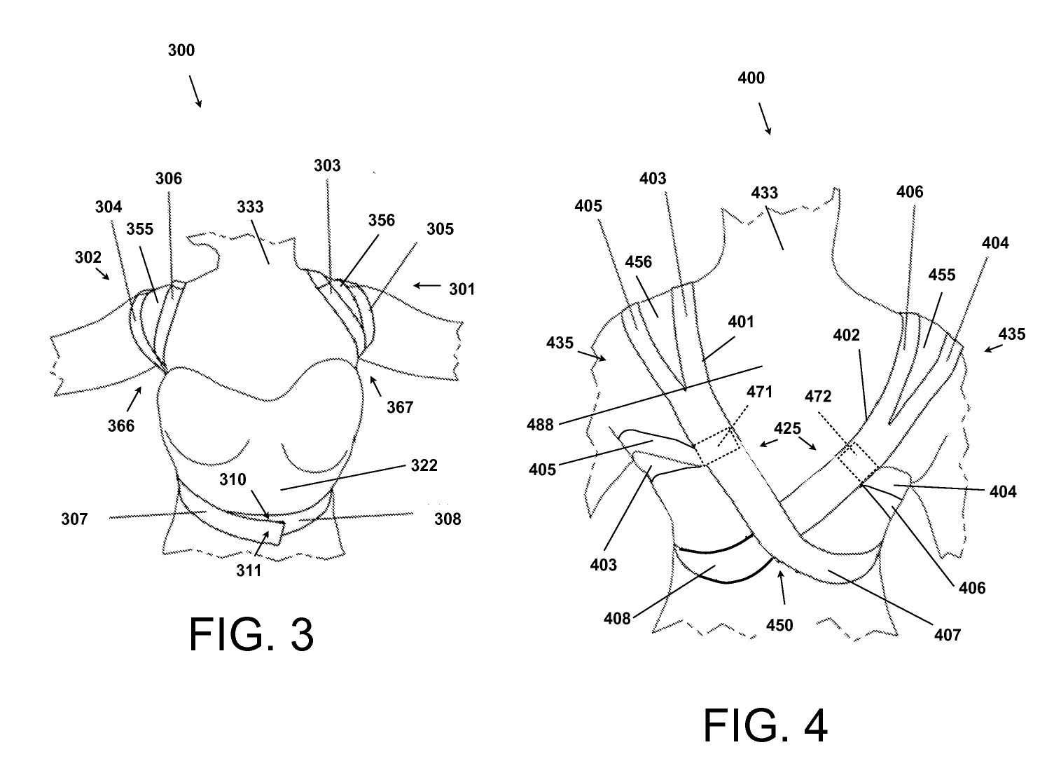

FIG. 3 is a frontal view showing one embodiment of the posture correcting assembly 300. Here, it is intended for the left-first-loop 303, and the left-second-loop 305 of the left strap 301 to hang from a left shoulder 356 of the user's 333 body. The right-first-loop 304, and the right-second-loop 306 of the right strap 302 to hang from a right shoulder 355 of the user's 333 body. The left-belt-band 307 wraps posteriorly around the user's body, the right-belt-band 308 wraps posteriorly around the user's body, and the left-attachment-point 311 and the right-attachment-point 310 interconnect at the anterior side or abdomen 322 of the user's body.

FIG. 4 is a view of the back of user 433 wearing the posture correcting assembly 400. Here, the right-first-loop 404, and the right-second-loop 406 of the right strap 402 loops around the right shoulder 455 of the user's 433 body, and simultaneously the left-first-loop 403, and the left-second-loop 405 of the left strap 401 loops around the left shoulder 456 of the user's 433 body. It is intended that both the left strap 401 and the right strap 402 cross 450 and overlap about the lower back 488 of a user's 433 body.

In this alternative embodiment, the left-first-loop 403, and the left-second-loop 405 are created by splitting the left strap 401 in two halves making the second end 435 a continuous form from left-belt-band 407. After the left-first-loop 403, and the left-second-loop 405 loop around a user's left shoulder 456, they are fixedly attached together 471, in a unitary form, in the middle portion 425 left-belt-band 407. Similarly, the right-first-loop 404, and the right-second-loop 406 are created by splitting the right strap 402 in two halves making the second end 435 a continuous form from right-belt-band 408. After the right-first-loop 404, and the right-second-loop 406 loop around a user's right shoulder 455, they are fixedly attached together 472, in a unitary form, in the middle portion 425 right-belt-band 408. Here, both left-belt-band 407 and right-band 408 cross one on top of the other, at middle point 450, in the back of a user's 433 body.

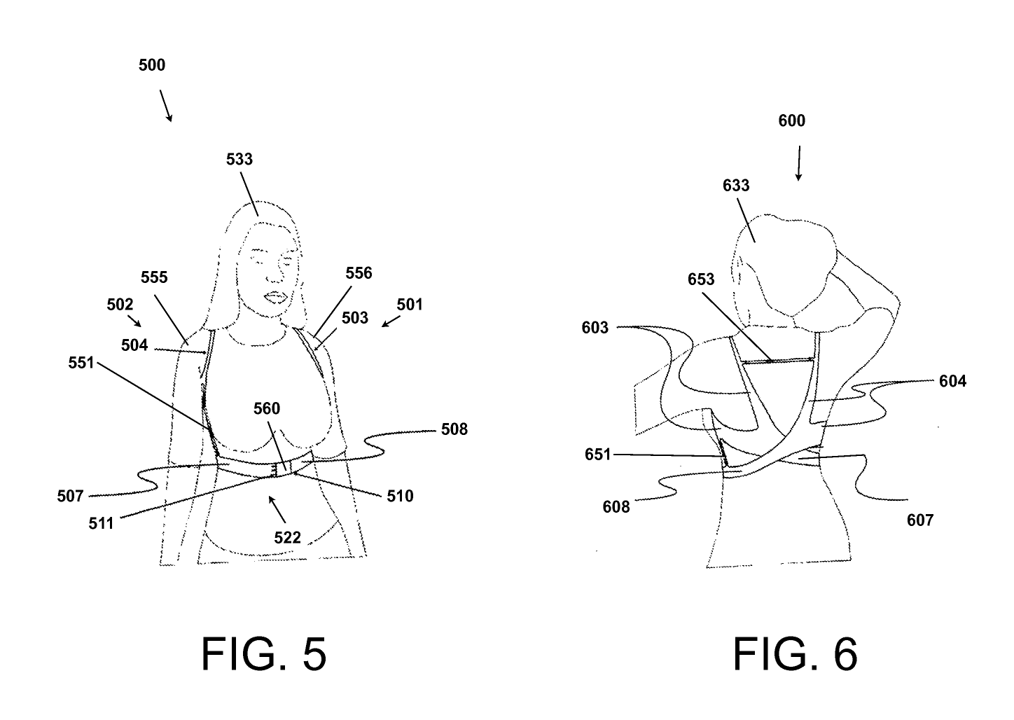

FIG. 5, is frontal view of a user 533 wearing an alternative embodiment of the posture correcting assembly 500. Here, the right strap 502, includes a single right-first-loop 504 looping around right shoulder 555 and similarly, the left strap 501 contains a single left-first-loop 503 looping around left shoulder 556. It is intended for both left and right attachment points 510 and 511 to demountably mate or couple by a buckle 560 together in front of the user a 533 bout abdomen 522.

As further shown in FIG. 5, the posture correcting assembly may further comprise at least one right-vertical-band 551, wherein the right-vertical-band 551 interconnects and demountably disposes right-first-loop 504 and left-belt-band 507. In some embodiments, the vertical band may be adjustable. In some embodiments, the vertical band 551 may be made of an elastic material, non-elastic material, rigid material, a semi-rigid material and more. The vertical band 551 may also include a buckle or some other means of attachment including a VELCRO fastening.

FIG. 6 is a graphical depiction of the back of a user 633 wearing the posture correcting assembly 500. As shown in FIG. 6, the posture correcting assembly 600 may further comprise at least one left-vertical-band 651 that interconnects and demountably disposes the left-first-loop 603 and the right band 608. The posture correcting assembly may also comprise at least one adjustable horizontal band 653, wherein the horizontal band 653 interconnects and demountably disposes the left-first-loop 603 and the a right-first-loop 604.

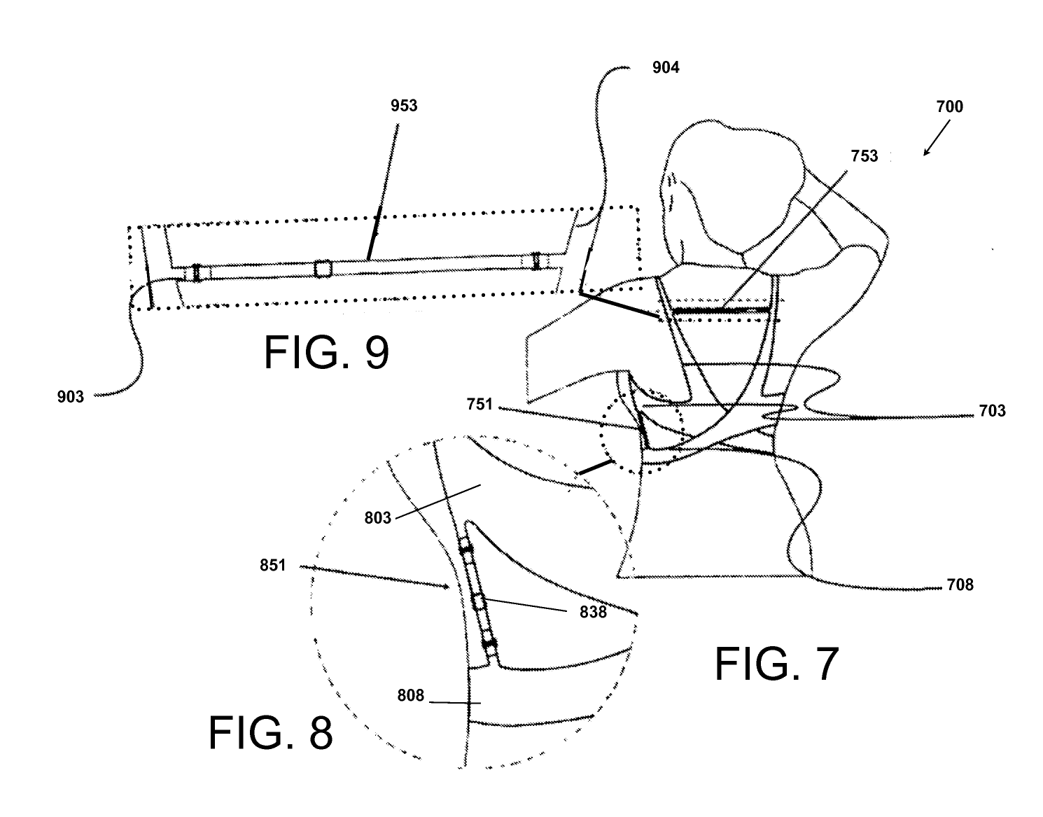

FIG. 7 is also a graphical depiction of the back of a user 633 wearing the posture correcting assembly 500. Here FIG. 8 and FIG. 9 should be viewed together with FIG. 7. FIG. 7 shows adjustable left-vertical-band 751 and adjustable horizontal band 753. FIG. 8 shows an exploded view of the left-vertical-band band 851. As shown, in this embodiment, the left-vertical-band 851 may be adjustable by movable clamp 838 and vertical-band 851 interconnects and demountably disposes the left-first-loop 803 and the right band 808. It is envisioned that in other embodiments, the vertical band 851 may be made of an elastic material, non-elastic material, rigid material, semi-rigid material and any combination thereof.

As shown in FIG. 9, an exploited view of the posture correcting assembly 700 of FIG. 7, it may also comprise at least one horizontal band 953. Horizontal band 953 interconnects and demountably disposes the right-first-loop 904 and the left-first-loop 903. The interconnection of the adjustable horizontal band 953 and the right-first-loop 904 and the left-first-loop 903 can be on either the posterior or anterior side of the user's body. In some embodiments, the at least one horizontal band 953 may be completely removable. In some embodiments, the at least one horizontal band 953 may be adjustable by a clamp or similar mechanism. It is envisioned that in other embodiments, the vertical band 953 may be made of an elastic material, non-elastic material, rigid material, semi-rigid material and any combination thereof.

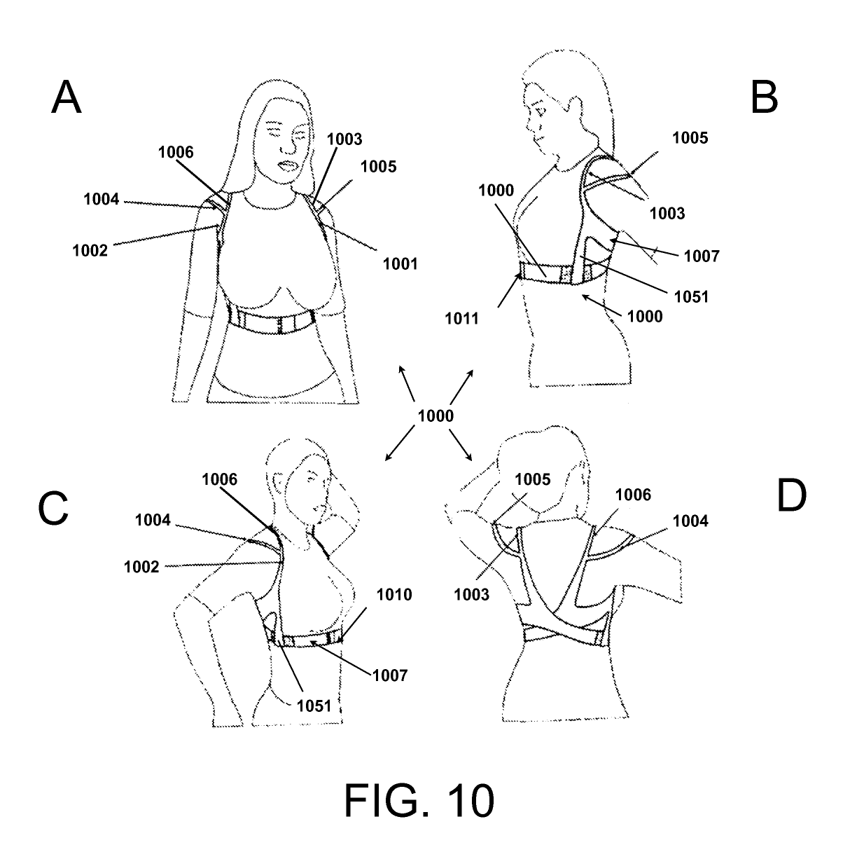

As shown in FIGS. 1-9, in some embodiments, the posture correcting assembly may comprise a single shoulder loop or multiple shoulder loops. Here in FIG. 10, are four graphical representation and multiple views of posture correcting assembly 1000 namely A, B, C and D. FIG. 10-A is a frontal view of a user wearing the posture correcting assembly 1000. Here an embodiment of the posture correcting assembly 1000 may further comprise a plurality loops, here, a right-first-loop 104, and a right-second-loop 106. The right-first-loop 104 may attach to the right-second-loop 106 in at least two places and hang from the user's arm at the arm-shoulder junction. The additional right-second-loop 106 provides added comfort by distributing the force put on the shoulder while wearing the posture correcting assembly. The additional right-second-loop 106 also functions to help keep the right-first-loop 104 in the correct position at all times. Furthermore, by having an additional loop, it provides for support and stability of the posture correcting assembly.

FIG. 10-B is a side view of a user wearing the posture correcting assembly 1000. In this alternative embodiment of the invention, the posture correcting assembly 1000 comprises a left strap 1001 that includes a left-first-loop 1003, a left-second-loop 1005, a left-belt-band 1007 and a left-attachment-point 1011, wherein the left-first-loop 1003, and the left-second-loop 1005 hangs from user's shoulder of a user's body. The left-belt-band 1007 wraps posteriorly around the wearer's body, and the left-attachment-point 1011 at the anterior of the user's body. Here posture correcting assembly 1000 further includes, at least one vertical band 1051 that interconnects the left-first-loop 1003 and the left-belt-band 1007. Here, the vertical band 1051 is demountably coupled to the left-belt-band 1007 by the use of VELCRO, but it is intended that other similar couplings can be used for the same purpose.

FIG. 10-C is a side view of a user wearing the posture correcting assembly 1000. In this alternative embodiment of the invention, the posture correcting assembly 1000 comprises a right strap 1002 that includes a right-first-loop 1004, a right-second-loop 1006, a right-belt-band 1008 and a left-attachment-point 1010, wherein the right-first-loop 1004, and the right-second-loop 1006 hangs from user's shoulder of a user's body. The right-belt-band 1008 wraps posteriorly around the wearer's body, and the left-attachment-point 1011 at the anterior of the user's body. Here posture correcting assembly 1000 further includes, at least one vertical band 1051 that interconnects the left-first-loop 1003 and the left-belt-band 1007. Here, the vertical band 1051 is demountably coupled to the left-belt-band 1007 by the use of VELCRO, but it is intended that other similar attachments can be used for the same purpose.

As shown here in FIG. 10-D, the back view of a user wearing the posture correcting assembly 1000. By having four shoulder loops, the left-first-loop 1003, provides the left-second-loop 1005 added comfort by distributing the force put on the shoulder while wearing the posture correcting assembly. Similarly, the right-first-loop 1004 also functions to help keep the right-second-loop 1006 in the correct position at all times.

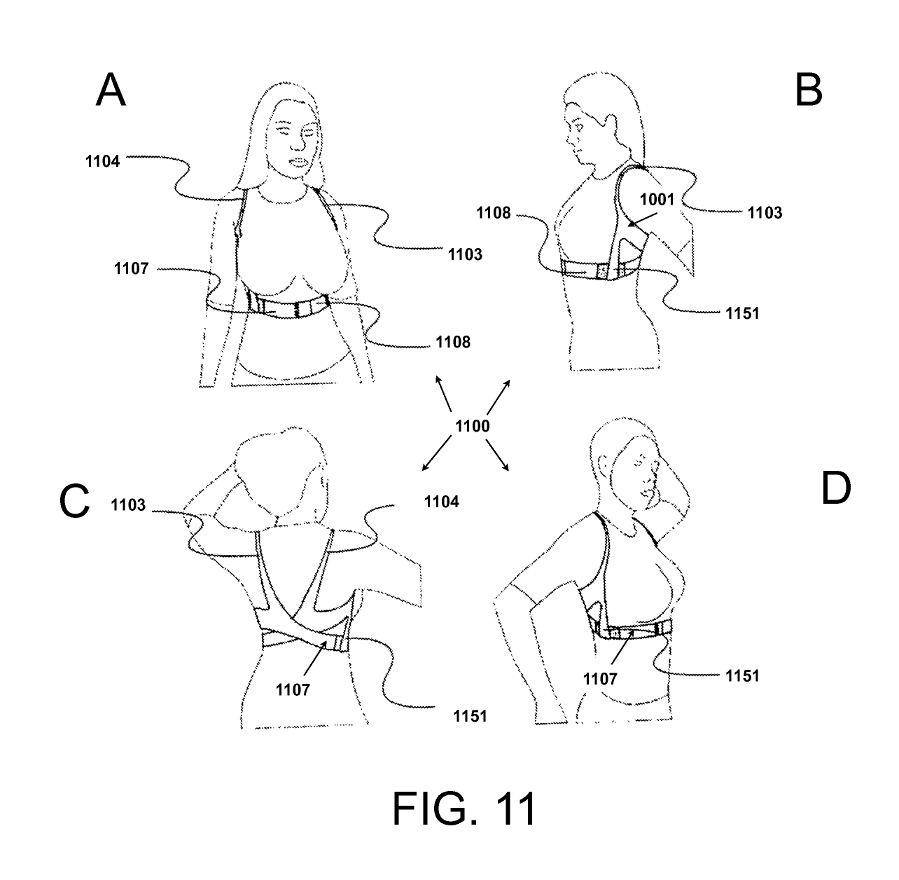

Here in FIG. 11, are four graphical representation and multiple views of posture correcting assembly 1100 namely A, B, C and D. As shown in FIGS. 11 A-D, in this embodiment, the posture correcting includes a single shoulder loop. FIG. 11-A is a frontal view of a user wearing the posture correcting assembly 1100. Here, the posture correcting assembly 1100 includes a left-first-loop 1103 and a right-first-loop 1104 that cross in the back of a user. The left-belt-band 1107 goes around the right of a user's waist, and similarly, the right-band 1108 goes the left of a user's waist to mutually intercept an interconnect in front of the user's abdomen.

FIG. 11-B is a side view of a user wearing the posture correcting assembly 1100. In this alternative embodiment of the invention, the posture correcting assembly 1100 includes a left strap 1101 that contains a left-first-loop 1103, a left-belt-band (not shown), wherein the left-first-loop 1103, hangs from user's shoulder of a user's left shoulder. And a vertical band 1151 that interconnects the left-first-loop 1103 and the right-belt-band 1108. Here, the vertical band 1151 is demountably coupled to the left-belt-band 1007 by the use of VELCRO, but it is intended that other similar attachments can be used for the same purpose.

As shown here in FIG. 11-C, the back view of a user wearing the posture correcting assembly 1100. As shown here, the posture correcting assembly 1100 includes a left-first-loop 1103 and a right-first-loop 1104 that cross in the back of a user. The vertical band 1151 that is demountably coupled to the left-belt-band 1007 by the use of VELCRO is also shown.

FIG. 11-D is a side view of a user wearing the posture correcting assembly 1000. In this embodiment of the invention, the posture correcting assembly 1100 includes vertical band 1151 that is demountably coupled to the left-belt-band 1007 by the use of VELCRO is also shown.

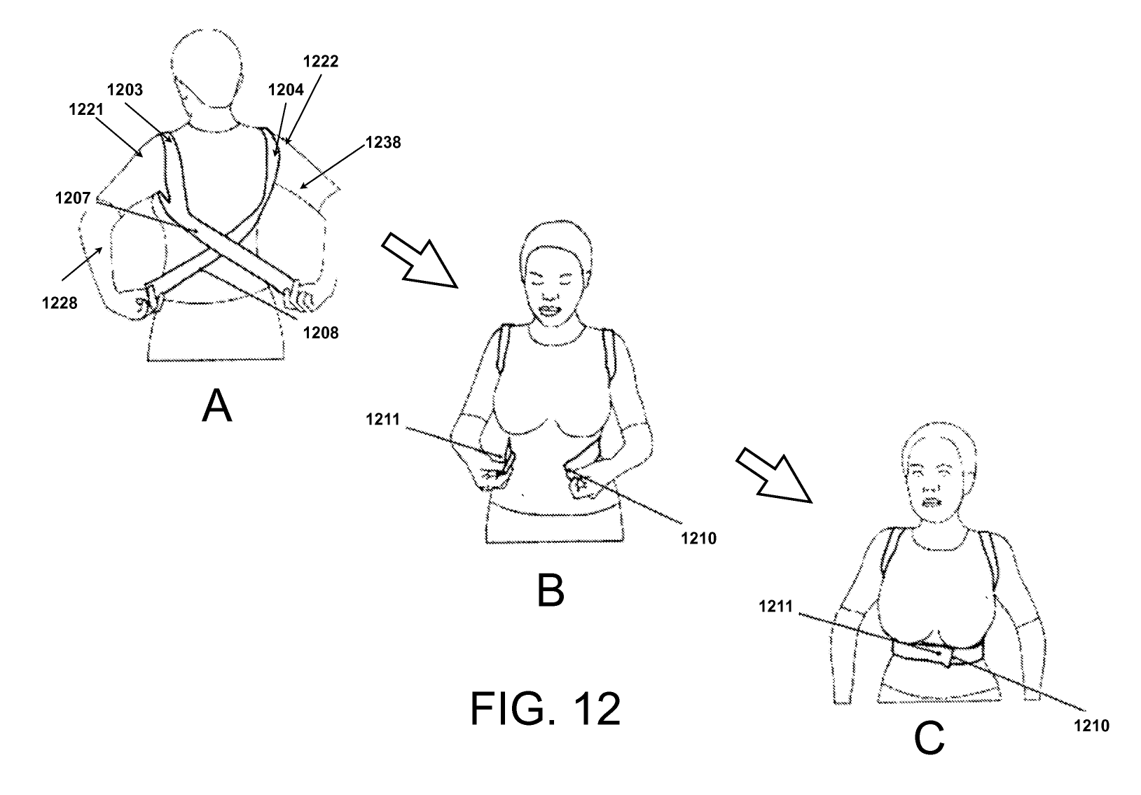

FIG. 12 are a series of three drawings A, B and C, showing the steps that a user takes to mount the posture correcting assembly described in previous figures an embodiments. As shown in FIGS. 12 A-C, the method for correcting posture of a user is depicted where a user obtains the left strap 101 and the right strap 102 of the posture correction assembly of the present invention and positions the straps on the body.

FIG. 12 A shows the method includes the steps of: inserting user's left arm through the a left-first-loop 1203 until the a left-first-loop 103 is in position around the user's left shoulder 1221, and then inserting user's second arm 1238 through the second loop 1204 until the second loop 1204 is in position around the user's second shoulder 1222. The method further includes extending the left-belt-band 1207 diagonally around the user's back toward the user's anterior side and extending the right-band 1208 diagonally around the user's back toward the user's anterior side, whereby the right-band 1208 and the left-belt-band 1207 crisscross over the user's mid or lower back.

FIG. 12 B and FIG. 12 C shows the method further including the step of attaching the left-attachment-point 1211 to the right-attachment-point 1210 on the anterior frontal side of the user about the user's abdomen by the use of VELCRO. Again, it is intended that other similar attachments can be used for the same purpose such as clamps, belt buckles, buttons etc. and more.

While the foregoing written description of the invention enables one of ordinary skill to make and use what is considered presently to be the best mode thereof, those of ordinary skill will understand and appreciate the existence of variations, combinations, and equivalents of the specific embodiment, method, and examples herein. The invention should therefore not be limited by the above described embodiment, method, and examples, but by all embodiments and methods within the scope and spirit of the invention.

It is to be appreciated that the Detailed Description section, and not the Abstract section, is intended to be used to interpret the claims. The Abstract section may set forth one or more but not all exemplary embodiments of the present invention as contemplated by the inventor(s), and thus, are not intended to limit the present invention and the appended claims in any way.

The present invention has been described above with the aid of functional building blocks illustrating the implementation of specified functions and relationships thereof. The boundaries of these functional building blocks have been arbitrarily defined herein for the convenience of the description. Alternate boundaries can be defined so long as the specified functions and relationships thereof are appropriately performed.

The foregoing description of the specific embodiments will so fully reveal the general nature of the invention that others can, by applying knowledge within the skill of the art, readily modify and/or adapt for various applications such specific embodiments, without undue experimentation, without departing from the general concept of the present invention. Therefore, such adaptations and modifications are intended to be within the meaning and range of equivalents of the disclosed embodiments, based on the teaching and guidance presented herein. It is to be understood that the phraseology or terminology herein is for the purpose of description and not of limitation, such that the terminology or phraseology of the present specification is to be interpreted by the skilled artisan in light of the teachings and guidance. The breadth and scope of the present invention should not be limited by any of the above-described exemplary embodiments, but should be defined only in accordance with the following claims and their equivalents.

We claim:

1. A posture correcting assembly, comprising:
a left strap defining a left-first-loop surrounding a left shoulder, the left strap further including:
a left-belt-band wrapping posteriorly around a user's body; and
a left-attachment-point; and
a right strap defining a right-first-loop surrounding a right shoulder, the right strap further including:
a right-belt-band wrapping posteriorly around a user's body; and
a right-attachment-point interconnect and is demountably disposed the left-attachment-point at the anterior side of a user's body.

2. The posture correcting assembly of claim 1, further comprising:
at least one horizontal band, wherein the horizontal band interconnects the left strap and the right strap.

3. The posture correcting assembly of claim 2, wherein:
the at least one horizontal band interconnects and demountably disposes left strap and the right strap on the anterior side of a user's body.

4. The posture correcting assembly of claim 2, wherein:
the at least one horizontal band interconnects and demountably disposes the left strap and the right strap on the posterior side of a user's body.

5. The posture correcting assembly of claim 2, wherein:
the at least one horizontal band is removable and adjustable.

6. The posture correcting assembly of claim 1, further comprising:
at least one left vertical band, wherein the left vertical band interconnects and demountably disposes the left-first-loop and the right-band.

7. The posture correcting assembly of claim 1, further comprising:
at least one right vertical band, wherein the right vertical band interconnects and demountably disposes the right-first-loop and the left-band.

8. The posture correcting assembly of claim 6, wherein:
the at least one left vertical band is removable and adjustable.

9. The posture correcting assembly of claim 7, wherein:
the at least one right vertical band is removable and adjustable.

10. The posture correcting assembly of claim 8, further comprising:
a left-second-loop and a right-second-loop.

11. A method for correcting posture of a user, the method comprising:
providing a posture correcting assembly, including:
a left strap defining a left-first-loop, the left strap further including: a left-belt-band, and a left-attachment-point; and
a right strap defining a right-first-loop, the right strap further including:
a right-belt-band; and a right-attachment-point;
inserting user's left arm through the left-first-loop until the left-first-loop is in position around the user's first shoulder;
inserting user's right arm through the right-first-loop until the right-first-loop is in position around the user's second shoulder;
extending the right-belt-band diagonally around the user's back toward the user's anterior side;
extending the left-belt-band diagonally around the user's back toward the user's anterior side, whereby the right-belt-band and the left-belt-band crisscross over the user's mid or lower back; and
attaching and demountably disposes the left-attachment-point to the right-attachment-point on the anterior side of the user.

17. A posture correcting assembly, comprising:
a first strap, the first strap comprising a first loop, a first band and a first attachment point;
a second strap, the second strap comprising a second loop, a second band and a second attachment point,
wherein the first loop hangs from a first shoulder of the user's body, the second loop hangs from a second shoulder of the users body, the first band wraps posteriorly around the wearer's body, the second band wraps posteriorly around the user's body, and the first attachment point and the second attachment point interconnect and demountably disposes at the anterior of the user's body;
at least one first vertical band, wherein the first vertical band interconnects and demountably disposes the first loop and the second band; and
at least one second vertical band, wherein the second vertical band interconnects and demountably disposes the second loop and the first band.

18. The posture correcting assembly of claim 17, further comprising:
at least one horizontal band, wherein the horizontal band interconnects and demountably disposes the first loop and the second loop.

19. The posture correcting assembly of claim 18, wherein:
the at least one horizontal band is adjustable.

20. The posture correcting assembly of claim 17, wherein:
the at least one first vertical band and the at least one second vertical band are adjustable
